(19) United States
(12) Patent Application Publication
(10) Pub. No.: US 2009/0280063 A1
(43) Pub. Date: Nov. 12, 2009

(54) NOVEL PEI-PEG GRAFT COPOLYMER COATING OF IRON OXIDE NANOPARTICLES FOR INFLAMMATION IMAGING

(75) Inventors: Amit Kulkarni, Clifton Park, NY (US); Brian Grimmond, Clifton Park, NY (US); Brian Christopher Bales, Niskayuna, NY (US); Chiaki Treynor, Palo Alto, CA (US); Daniel E. Meyer, Niskayuna, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company, Schenectady, NY (US)

(21) Appl. No.: 12/117,873

(22) Filed: May 9, 2008

(51) Int. Cl. A61K 49/06; A61K 9/14; A61P 43/00

(52) U.S. Cl. 424/9.3; 424/501

(57) ABSTRACT

A nanostructure includes a nanoparticle core (110) and a ligand (120) bonded to the nanoparticle core (110). The ligand includes a linking group (130) having a first end bonded to a polyethylene imine (PEI) polymer (140) and a second end bonded to the nanoparticle core (110) and a polyethylene glycol (PEG) polymer (150) grafted to the PEI polymer (140). Methods for making these nanostructures and their use in magnetic resonance imaging and management of inflammatory conditions are provided.

### BACKGROUND

A recurring challenge in developing nanoparticle-based imaging agents for magnetic resonance imaging is controlling surface charge of the MR active nanoparticles without significantly effecting the overall nanoparticle size. Nanoparticles typically have very high surface energies and as a result, they form aggregates quite easily. Nanoparticles prepared in the absence of a surface stabilizing ligands readily form aggregates in solution. This aggregation can be prevented through the binding of ligands to the surface of the nanoparticle. These ligands may prevent aggregation through either steric or electrostatic repulsions.

Typically, the surface charge of nanoparticles can be varied through the use of different stabilizing ligands that bind to the surface of the nanoparticle. Different charged stabilizing ligands are often different lengths and as a result, different ligands effect the overall size of the nanoparticle.

Known nanoparticle-based agents have included iron oxide cores stabilized by biocompatible coatings such as dextran, starch, or carbohydrate. Typically, the iron oxide core diameter ranges from about 3 to about 10 nm and the diameter of the core and coating combined ranges from about 10 to about 100 nm. Known nanostructures, such as Feridex™ and Resovist™, are negatively charged and have a short blood residence time (human blood half-life of less than 1 hour) precluding them from accessing tissue with slow uptake. Hence, agents with a short blood residence time are ill suited for imaging such tissue and subendothelial spaces, for example, the intima of blood vessels. Existing superparamagnetic particle contrast agents also suffer from various disadvantages, such as wide size distribution, agglomeration, instability, and toxicity.

Combidex™, with a dextran coating and a diameter of 15-30 nm, has been evaluated for magnetic resonance imaging in a variety of animal disease models as well as in humans. Due to its small size, Combidex™ has a long blood residence time (human blood half-life between 24-36 hours.

Needs remain for nanostructures of appropriate solubility, biocompatibility, size, and coating characteristics that are capable of being efficiently internalized by inflammatory response cells and trafficked to the site of inflammation for use in imaging inflamed tissue. Given that biodistribution properties of nanoparticles designed for in vivo use are strongly influenced by the overall nanoparticle size and the surface charge, the ability to vary the nanoparticle surface charge without changing the nanoparticle size is desirable as nanoparticles of the same size with varying surface charges allow for the effects of surface charge on nanoparticle biodistribution to be decoupled from size effects.

### SUMMARY

In some aspects, embodiments disclosed herein provide a nanostructure including: (1) an inorganic nanoparticle core; (2) a ligand bonded to the nanoparticle core, the ligand including a linking group having a first end bonded to a polyethylene imine (PEI) polymer; and a second end bonded to the nanoparticle core; and (3) a polyethylene glycol (PEG) polymer grafted to the PEI polymer.

In other aspects, embodiments disclosed herein provide a method of making the these nanostructures. The method includes reacting a nanoparticle core with a PEI-PEG graft having a linking group. The linking group has a functional group capable of reaction with the nanoparticle core and is selected from the group consisting of a carboxylate, a sulfonate, a phosphate, and a trialkoxysilane.

In yet other aspects, embodiments disclosed herein provide a method of imaging an inflammatory condition in a mammal. The method includes introducing into the mammal the above described nanostructures into inflammatory cells in vivo or ex vivo, permitting the inflammatory cells to migrate to inflamed tissue, and imaging the inflamed tissue using magnetic resonance.

Advantageously, the nanostructures disclosed herein may be useful as magnetic resonance imaging agents that can be used in visualization and management of inflammatory conditions.

The foregoing has outlined rather broadly the features of the present disclosure in order that the detailed description that follows may be better understood. Additional features and advantages of the invention will be described hereinafter, which form the subject of the claims of the invention.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 shows nanostructure 100 having a polyethylene imine (PEI)-polyethylene glycol (PEG) graft copolymer attached to a nanoparticle core via a silane linkage.

FIG. 2 shows nanostructure 200 having polyethylene imine (PEI)-polyethylene glycol (PEG) graft copolymer attached to a nanoparticle core via a silane linkage using a PEG-350 graft.

FIG. 3 shows nanostructure 300 having a polyethylene imine (PEI)-polyethylene glycol (PEG) graft copolymer attached to a nanoparticle core via a silane linkage using a PEG-750 graft.

FIG. 4 shows nanostructure 400 having polyethylene imine (PEI)-polyethylene glycol (PEG) graft copolymer attached to a nanoparticle core via a silane linkage using a carboxylate-terminated PEG graft.

FIGS. 5A-C show the ¹H NMR spectra of PEI plus MeO-PEG-mesylate before (A) and after (B) reaction. MeO-PEG-mesylate is derived from PEG-350.

### DETAILED DESCRIPTION OF THE INVENTION

In the following description, specific details are set forth such as specific quantities, sizes, etc. so as to provide a thorough understanding of embodiments of the present invention. However, it will be obvious to those skilled in the art that the present invention may be practiced without such specific details. In many cases, details concerning such considerations and the like have been omitted inasmuch as such details are not necessary to obtain a complete understanding of the present invention and are within the skills of persons of ordinary skill in the relevant art.

Referring to the drawings in general, it will be understood that the illustrations are for the purpose of describing a particular embodiment of the invention and are not intended to limit the invention thereto.

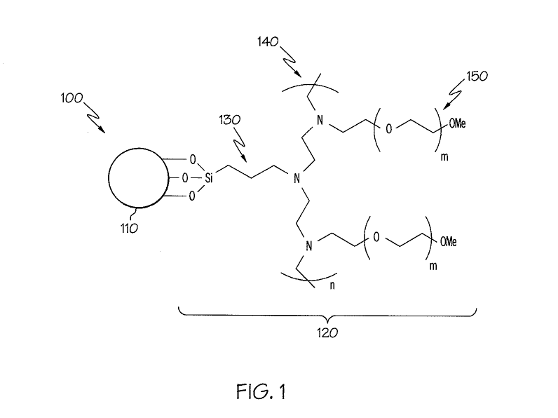

In some embodiments, the present disclosure relates to nanostructured materials that may be useful as imaging agents. With reference to FIG. 1, a nanostructure 100 for such purposes includes an inorganic nanoparticle core 110. Inorganic nanoparticle core 110 is generally any material that can serve as a magnetic resonance (MR) active entity (i.e. a signal source for MR) and that can serve as a platform for chemical modification to affect the overall charge/size/polarity of nanostructure 100. Charge and size of nanostructure 100 are typically influenced by the ligands bound to inorganic nanoparticle core 110. In the example of FIG. 1, ligand 120 is includes a linking group 130 which attaches at a first end to a polyethylene imine (PEI) polymer 140. A second end of linking group 130 attaches to nanoparticle core 110. Finally, the PEI polymer has grafted about it's outer portion polyethylene glycol (PEG) polymer 150. The PEG appendages provide substantial solubility to nanostructure 100. Although in FIG. 1 PEI is linked to nanoparticle core 110 via a linking group having a silane moiety, the silane linkage to nanoparticle core 110 is but one exemplary embodiment as elaborated further hereinbelow.

While most of the terms used herein will be recognizable to those of skill in the art, the following definitions are nevertheless put forth to aid in the understanding of the present disclosure. It should be understood, however, that when not explicitly defined, terms should be interpreted as adopting a meaning presently accepted by those of skill in the art.

“Nanoscale,” as defined herein, generally refers to dimensions below 1 μm.

“Nanostructures,” as defined herein, generally refer to structures that are nanoscale in at least one dimension. Nanostructures disclosed herein may be useful in, among other applications, medical imaging procedures such as magnetic resonance imaging.

As used herein the terms “zeta potential,” “surface potential,” and “surface charge” and the abbreviation “ζ” refers to a measurement of the electrostatic potential near the surface of the particle. As the zeta potential is affected by the solvent and ionic strength of the solvent, all zeta potential values reported herein are measured using 10 mM aqueous NaCl as the solvent unless otherwise indicated. Thus, the cationic nanostructures of the invention display a zeta potential of about between about 0 and about +60 mV.

As used herein, the terms “hydrodynamic diameter,” “hydrodynamic size,” and the abbreviation “D_{H}” refer to the diameter of spherical particle that would have a diffusion coefficient equal to that of the nanoparticle as measured by dynamic light scattering (DLS). D_{H} values may vary depending on the medium in which the agent being measured is dispersed. Thus, unless otherwise indicated, the D_{H} values described herein were measured using DLS where the agent is dispersed in 150 mM aqueous NaCl.

As used herein, “quaternization” or “quaternized” nitrogen refers to nitrogen atoms forming four bonds, which may be covalent or ionic for example. Thus, the quaternization of nitrogen results in a formal positive charge on nitrogen. The quaternization may be reversible through adjustment of pH, thus affecting the degree of protonation of available nitrogen atoms in the PEI portion of the polymer bound to the inorganic core structure.

Inorganic nanoparticle core 110 may be any material that provides a magnetic resonance signal and is capable of chemical modification to alter the size and charge of the nanostructure. Such structures may include paramagnetic materials, superparamagnetic materials, and the like. Superparamagnetic inorganic nanoparticle cores may include (1) iron oxides (such as hematite, ferrite, and magnetite) (2) a mixed spinnel ferrite having a the general formula MFe₂O₄, where M is a metal, including without limitation, manganese, cobalt, copper, nickel, and magnesium; and (3) combinations thereof. In some embodiments, the inorganic nanoparticle core comprises a superparamagnetic iron oxide (SPIO) agent. Nanostructures may include superparamagnetic iron oxide crystalline structures that have the general formula [Fe₂⁺O₃]ₓ[Fe₂⁺O₃(M²⁺O)]₁₋ₓ where 1≧x≧0. M²⁺ may be a divalent metal ion such as iron, manganese, nickel, cobalt, magnesium, copper, or a combination thereof. When the metal ion (M²⁺) is ferrous ion (Fe²⁺) and x=0, the Nanostructure is magnetite (Fe₃O₄), and when x=1, the Nanostructure is maghemite (Fe₂O₃, γ-Fe₂O₃).

In general, superparamagnetism occurs when crystal-containing regions of unpaired spins are sufficiently large that they can be regarded as thermodynamically independent, single domain particles called magnetic domains. These magnetic domains display a net magnetic dipole that is larger than the sum of its individual unpaired electrons. In the absence of an applied magnetic field, all the magnetic domains are randomly oriented with no net magnetization. Application of an external magnetic field causes the dipole moments of all magnetic domains to reorient resulting in a net magnetic moment. In some embodiments, nanostructures demonstrate a spinel crystalline structure as shown by transmission electron microscope (TEM) analysis.

Inorganic nanoparticle core 110 may be roughly spherical in shape having a diameter ranging from about 1 nm to about 100 nm in one embodiment and from about 1 nm to about 10 nm in another embodiment. One skilled in the art will recognize that irregularities deviating from perfect spherical geometry are typical for the inorganic nanoparticle core.

In accordance with various embodiments, the ligand bound to the inorganic nanoparticle core includes the PEG-grafted PEI ligand 120 as shown in FIG. 1. In some embodiments, however, the outer graft polymer on the PEI polymer may include other hydrophilic ligands, ideally with the proviso that the chosen ligand imparts biocompatibility to the overall structure. Examples of chemical structures that may impart biocompatibility include, without limitation, derivatives of PEG, polyvinylpyrrolidone, poly L-lysine, and the like. Biocompatibility includes solubility, generally in water, as well as non-toxicity.

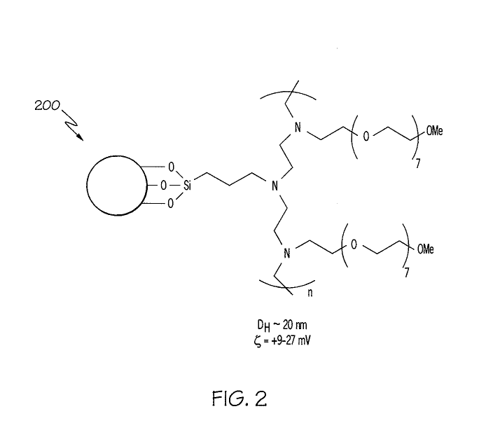

It is desirable to be able to vary the charge of the overall nanostructure without appreciably changing its size in order to effectively study the biodistribution of these imaging agents. FIG. 2 shows a nanostructure 200 having a SPIO core and PEG-350 grafted to the PEI polymer. The hydrodynamic diameter of the construct is approximately 20 nm over a variety of zeta potentials ranging from about 9 mV to about 27 mV. The charge on nanostructure 200 may be influenced by the protonation state of the amine nitrogens of the PEI polymer, for example. In principle is it also possible to use other reagents to quaternize nitrogen such as Lewis acids and/or reaction with alkylating agents to generate tetra-alkyl ammonium salts. One skilled in the art will recognize, however, that a proton source is relatively small and would likely have the least impact on the hydrodynamic diameter over varied zeta potential. The hydrodynamic diameter may be influenced by the length of the PEG graft in addition to the degree of grafting at available nitrogen sites.

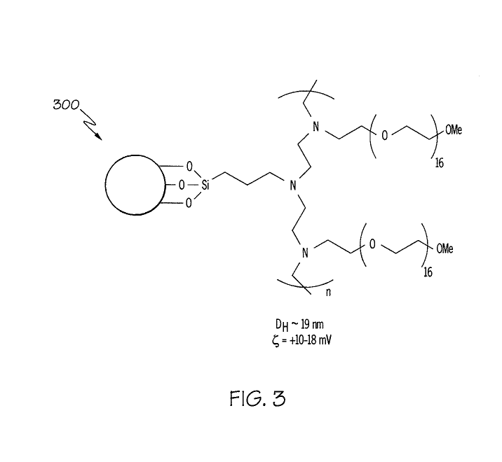

As shown in FIG. 3 nanostructure 300 has a SPIO core with the longer PEG-750 graft to the PEI polymer. The hydrodynamic diameter of the construct is approximately 19 nm over a variety of zeta potentials ranging from about 10 mV to about 18 mV. In some embodiments, the PEG polymer grafted to the PEI polymer has a molecular weight ranging from between about 350 Daltons to about 5000 Daltons. In some embodiments the ligand can include biocompatible polymers other than PEG polymer. For example, homo and co-polymers that may prove useful as part of the ligand include polyvinylpyrrolidone, poly L-lysine, and the like. The PEI polymer itself may have a molecular weight ranging from between about 800 Daltons to about 1600 Daltons. In addition to factors such as pH the size of the PEI polymer affects the overall charge based on the number of available nitrogen atoms available for quaternization.

The PEI portion of the nanostructure may be bonded to the inorganic nanoparticle core by a variety of functional groups, including for example carboxylates, sulfonates, phosphates, and silanes (as exemplified in FIGS. 1 and 2). Other covalent bonding motifs engaging the functional groups of a given inorganic nanoparticle core will be readily recognized by one of ordinary skill in the art. For example, with pendant OH groups on the inorganic nanoparticle core, sulfinate, sulfite, phosphinate, phosphonite, phosphonate, thiosulfate and even ether linkages are also possible. The functional group that links to the inorganic nanoparticle core and the PEI shell may be an interceding group that connects the two portions. The interceding group can vary substantially in structure, although one skilled in the art will appreciate the benefits of a minimal size for such linking group so that the overall properties of the nanostructure are not adversely affected.

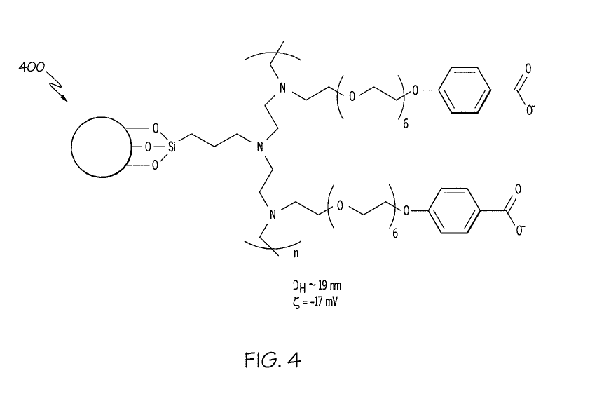

In some embodiments the nanostructure may include a ligand having a PEG polymer with a negatively charged terminal functional group, such as nanostructure 400 shown in FIG. 4. The degree of negative charge may be controlled by pH in this example as well in a manner analogous to the protonation state/quaternization of the amines in structures 100, 200, and 300, except that the degree of deprotonation of the carboxylic acid functional groups will be proportional to the negative zeta potential. In principle, structure 400 may also operate in a positive zeta potential regime at low pH where the carboxylate groups are substantially fully protonated and the nitrogen atoms of the PEI polymer are also quaternized.

By varying the protonation state of nitrogen in PEI of structures 100, 200, and 300 or the deprotonation state of carboxylate structure 400 one can introduce a non-zero surface charge on the nanostructure in a range from between about −50 mV to about +50 mV. In some embodiments, the surface charge has a non-zero surface charge in a range from between about −25 to about +25 mV. In further embodiments, the surface charge in a range from between about −5 mV to about −15 mV, and in yet further embodiments the surface charge is in a range from +5 mV to about +15 mV. One skilled in the art will recognize the value of being able to tune the surface charge depending on factors such as tissue-type being targeted, blood half-life, rate of cellular uptake, and clearance pathway.

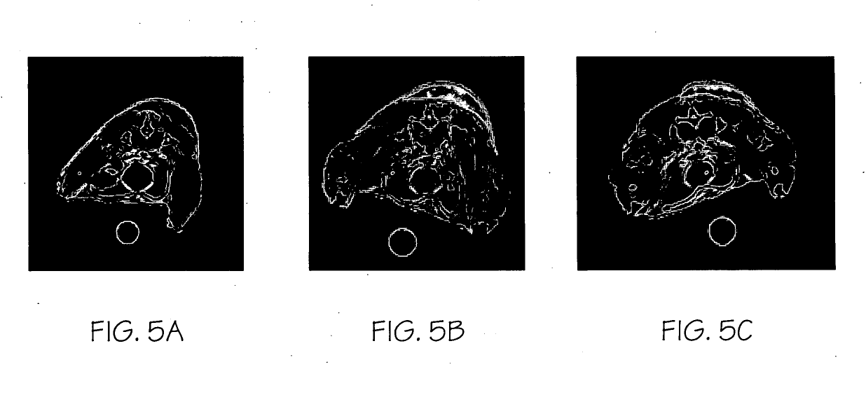

The present disclosure provides a method of making the above described nanostructures. The method broadly includes reacting a nanoparticle core with a PEI-PEG graft having a linking group, wherein the linking group has a functional group capable of reaction with the nanoparticle core. Consistent with the above discussion the functional group for attachment to the core may be a carboxylate, a sulfonate, a phosphate, or a trialkoxysilane. In one embodiment the PEI-PEG graft is preformed and the graft subsequently loaded onto the nanoparticle core (typically a SPIO core). In other embodiments, the PEI polymer may be attached first to the core and then the PEG grafted thereafter. FIGS. 5A-C show the ¹H NMR progress in the formation of a PEI-PEG graft copolymer which may be useful in subsequent attachment to a SPIO core, for example. The reaction follows the disappearance of the mesylate methyl group resonance. FIG. 5A shows a T2*-weighted MR image before injection of PEG-750 PEI-Silane agent. FIG. 5B shows a T2*-weighted MR image 2 hours after injection of PEG-750 PEI-Silane agent. FIG. 5C shows a T2*-weighted MR image 24 hours after injection of PEG-750 PEI-Silane agent. The granuloma location is indicated by the arrow head in FIGS. 5B and 5C. The arrows indicate the location of notable T2* darkening in the margin between granuloma and muscle following agent administration.

Finally, the present disclosure also provides a method of imaging an inflammatory condition in a mammal that includes introducing into the mammal (the mammal may be a human subject, for example) the nanostructures described hereinabove into inflammatory cells in vivo or ex vivo. The method includes permitting the inflammatory cells to migrate to inflamed tissue and imaging the inflamed tissue using magnetic resonance. In conjunction with the visualization methods, one can integrate management of the inflammatory condition.

The nanostructures described herein may be dispersed in physiologically acceptable carrier to minimize potential toxicity. Thus, the nanostructures of may be dispersed in a biocompatible solution with a pH of about 6 to about 8. In some embodiments, the nanostructure is dispersed in a biocompatible solution with a pH of about 7 to about 7.4. In other embodiments, the nanostructure is dispersed in a biocompatible solution with a pH of about 7.4.

The nanostructures may be combined with additives that are commonly used in the pharmaceutical industry to suspend or dissolve the compounds in an aqueous medium, and then the suspension or solution can be sterilized by techniques known in the art. The nanostructures or their pharmaceutically acceptable salts can be administered to a subject (including human subjects) in a variety of forms adapted to the chosen route of administration. Thus, the nanostructures may be introduced topically (i.e., by the administration to the tissue or mucus membranes), intravenously, intramuscularly, intradermally, and/or subcutaneously. Forms suitable for injection include sterile aqueous solutions or dispersions and sterile powders for the preparation of sterile injectable solutions, dispersions, liposomal, or emulsion formulations. In all cases, the form should be sterile and sufficiently fluid to enable administration by a syringe. Forms suitable for inhalation use include nanostructures dispersed in a sterile aerosol. Forms suitable for topical administration include creams, lotions, ointments, and the like.

In some embodiments, the nanostructures are concentrated to conveniently deliver a preferred amount of the nanostructures to a subject and packaged in container in the desired form. Thus, in some embodiments the nanostructure is dispensed in a container dispersed in physiologically acceptable solution, that conveniently facilitates administering the nanostructure in concentrations of about 0.1 mg of Fe content of the agent per kg body weight of the subject (i.e., 0.1 mg Fe/kg bw) to about 50 mg Fe/kg bw. In other embodiments, the nanostructure is packaged in a manner that conveniently facilitates administration of the nanostructure in concentrations of about 0.5 mg Fe/kg bw to about 2.5 mg Fe/kg bw.

In some embodiments, the disclosed nanostructures may be administered directly to the subject in a variety of ways including topically, intravascularly, intramuscularly, or interstitially. In some embodiments, about 0.1 mg Fe/kg to about 50 mg Fe/kg of Nanostructure is administered to the subject. In other embodiments, about 0.5 mg Fe/kg to about 2.5 mg Fe/kg of agent is administered to the subject. Similarly, inflammatory response cells containing of the disclosed nanostructures may be administered to the subject in a variety of ways including intravascularly, intramuscularly, or interstitially.

In some embodiments, the target tissue is imaged less than or approximately 3 hours after administering the nanostructures or inflammatory response cells containing the nanostructures. In alternative embodiments, the target tissue is imaged less than or approximately 24 hours after administering to the subject the nanostructures or inflammatory response cells containing nanostructures. In other embodiments, target tissue is imaged less than or approximately 5 days after administering to the subject the nanostructures or inflammatory response cells containing nanostructures.

In another series of embodiments, the present invention provides for methods of imaging conditions associated with inflammatory response cells infiltration and accumulation using the nanostructures. The nanostructures may be introduced into inflammatory response cells ex vivo and subsequently introduced into the subject. Thus, the inflammatory response cells may be withdrawn from the subject, the nanostructure introduced into the inflammatory response cells, and the inflammatory response cells containing the nanostructure are administered to subject prior to imaging. The step of introducing the nanostructures into the inflammatory response cells may optionally include the step of separating the inflammatory response cells using magnetic beads, density agents and/or centrifugation, for example. In certain embodiments, the inflammatory response cells comprise monocytes circulating in the blood, macrophage cells in tissue, dendritic cells (DCs), polynuclear monocytes (PNMs), eosinophils, neutrophils, and T cells.

The methods of managing conditions associated with inflammatory response cell infiltration and accumulation may include imaging the target tissue before, after, or both before and after treating the subject to reduce inflammation. Thus, the disclosed methods of managing conditions associated with inflammatory response cell infiltration and accumulation may include (a) imaging the target tissue to obtain base-line or diagnostic information about an inflammatory condition, (b) treating the subject, and (c) imaging the subject a one or more times to obtain further information about the inflammatory condition. A medical professional may opt not to image the subject both before and after treatment, relying on other techniques to initially characterize the inflamed tissue or subsequently assess the inflamed tissue. Thus, in an alternative embodiment, the methods of managing conditions associated with inflammatory response cell infiltration and accumulation includes treating an inflammatory condition that was identified by a technique other than magnetic resonance and imaging the target issue subsequent to treatment. Likewise, in another alternative embodiment, the disclosed methods of managing conditions associated with inflammatory response cell infiltration and accumulation may include imaging a subject or target tissue to obtain information about an inflammatory condition followed by treating the inflammatory condition without subsequently re-imaging the target tissue.

When the disease management is directed to determining the efficacy of a treatment, the methods comprise imaging the tissue of interest before administration of a treatment to obtain a pre-treatment assessment, followed by administration of the treatment and imaging the tissue of interest one or more times subsequent to the treatment to obtain a post-treatment assessment of the tissue of interest. The pre-treatment assessment and the post-treatment assessment(s) may be compared to determine whether the reduced inflammation or otherwise ameliorated the symptoms of the condition associated with inflammatory response cells infiltration and accumulation. The methods of determining the efficacy of a treatment may further comprise deciding whether to cease a particular treatment, as well as decisions to increase the frequency, intensity, and/or dose of a treatment based on the comparison of the pre- and post-treatment assessments.

When the disease management includes treatments that are localized to the inflamed tissue rather than a holistic or systemic administration of treatment (e.g., surgical or radiological intervention), the disease management methods may include determining the spatial localization of the inflamed tissue to define the specific area to be treated (e.g., excised or irradiated).

The methods described hereinabove can be used in treatments to decrease inflammation before, after, or before and after imaging the inflammatory condition. The imaging results can be used in the management of the inflammatory condition. Inflammatory conditions of particular interest are those associated with macrophage accumulation, including, without limitation, autoimmune conditions, vascular conditions, neurological conditions, and a combination thereof.

Experimental Examples

The following examples are provided to more fully illustrate some of the embodiments of disclosed hereinabove. It should be appreciated by those of skill in the art that the techniques disclosed in the examples which follow represent techniques that constitute exemplary modes for its practice. However, those of skill in the art should, in light of the present disclosure, appreciate that many changes can be made in the specific embodiments that are disclosed and still obtain a like or similar result without departing from the spirit and scope of the invention.

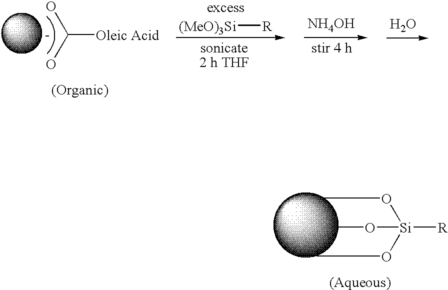

### Synthesis of 5 nm SPIO Nanoparticles.

A 25 mL, 3-neck Schlenk flask was fitted with a condenser, stacked on top of a 130 mm Vigreux column, and a thermocouple. The condenser was fitted with a nitrogen inlet and nitrogen flowed through the system. The Schlenk flask and Vigreux column were insulated with glass wool. Trimethylamine-N-oxide (Aldrich, 0.570 g, 7.6 mmol) and oleic acid (Aldrich: 99+%, 0.565 g, 2.0 mmol) were dispersed in 10 mL of dioctylether (Aldrich: 99%). The dispersion was heated to 80° C. at a rate of about 20° C./minutes. Once the mixture had reached ˜80° C., 265 μL of Fe(CO)₅ (Aldrich: 99.999%, 2.0 mmol) was rapidly injected into the stirring solution through the Schlenk joint. The solution turned black instantaneously, with a violent production of a white “cloud.” The solution rapidly heated to ˜120-140° C. Within 6-8 minutes the reaction pot cooled to 100° C. at which it was kept and stirred for 75 minutes. After stirring at ˜100° C. for 75 minutes, the temperature was increased to ˜280° C. at a rate of about 20° C./min. After the solution stirred for 75 minutes, the heating mantel and glass wool were removed to allow the reaction to return to room temperature.

Characterization

Hydrodynamic diameter was measured via dynamic light scattering using 150 mM NaCl as the solvent. The purified SPIO solution was diluted with 150 mM NaCl and passed through a 100 nm filter prior to DLS analysis using a Brookhaven ZetaPALS. Zeta potential was measured using a Brookhaven ZetaPALS after diluting the SPIO solution 14× with H₂O (final solution (10 mM NaCl) and passing the diluted SPIO solution through a 100 nm filter.

| | PEG:Si | | Zeta potential |
|---|---|---|---|
| Shell | (synth) | D_{H} (nm) | (mV) |
| PEI-Silane | 0 | ~16 | ~ +40 |
| PEG-350 PEI- | 3.6 | 21.4 ± 0.1 | +26.9 ± 9.2 |
| Silane (A) | | | |
| PEG-350 PEI- | 7.3 | 17.3 ± 5.6 | +9.1 ± 4.5 |
| Silane (B) | | | |
| PEG-750 PEI- | 3.6 | 15.9 ± 2.7 | +18.3 ± 2.6 |
| Silane (A) | | | |
| PEG-750 PEI- | 7.3 | 21.6 ± 3.5 | +14.9 ± 2.7 |
| Silane (B) | | | |
| PEG-750PE1- | 14.6 | 18.4 ± 1.1 | +10.4 ± 3.0 |
| Silane (C) | | | |

In Vivo Experiments

Granulomas were induced in female Swiss Webster mice by subcutaneous injection of 0.1 mL of a 1% carrageenan suspended in sterile physiologic phosphate-buffered saline. The injection site was dorsally located 1 cm superior to the base of tail. SPIO contrast agent was then injected intravenously via the tail vein in physically restrained mice between 2 and 7 days following granuloma induction. SPIO agent was in physiologic saline at a concentration of 5 mg Fe/mL, and was sterile filtered prior to injection and tested for the presence of endotoxin. The agent was dosed at 20 mg Fe/kg body weight.

The mice were imaged prior to injection of SPIO contrast agent, and again at ˜24 hrs post injection of the agent. Mice were imaged on a clinical 1.5 T GE Signa MR scanner using a custom-built, 3.2 cm solenoid transmit/receive RF coil. The mice were anesthetized using 2% isoflurane in oxygen by nose cone using a commercial anesthesia machine designed for rodents. For each of 2 pulse sequences, 13 transaxial 1 mm image slices were collected to obtain full coverage of the granuloma. The pulse sequence parameters were as follows:
T1-weighting: 2D Spin Echo, TE 13, TR 320, matrix 256×192, FOV 5, phase FOV 0.75, thickness 1.0, NEX 3, BW 22.73.
T2*-weighting: 2D Gradient Echo, TEI 9.8, TE2 25, TR 650, flip ang 45, matrix 256×192, FOV 5, phase FOV 0.75, slice thickness 1.0, NEX 2, BW 15.63.

It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present invention, but they are not essential to its practice. It is therefore to be understood that the invention may be practiced otherwise than as specifically described without actually departing from the spirit and scope of the present invention as defined by the appended claims.

What is claimed is:

1. A nanostructure comprising:
a nanoparticle core;
a ligand bonded to the nanoparticle core, the ligand comprising:
a linking group having:
a first end bonded to a polyethylene imine (PEI) polymer; and
a second end bonded to the nanoparticle core; and
a polyethylene glycol (PEG) polymer grafted to the PEI polymer.

2. The nanostructure of claim 1, wherein the nanoparticle core comprises superparamagnetic iron oxide.

3. The nanostructure of claim 1, wherein the nanoparticle core has a diameter ranging from about 1 nm to about 100 nm.

4. The nanostructure of claim 1, wherein the nanoparticle core has a diameter of about 1 nm to about 10 nm.

5. The nanostructure of claim 1, wherein the second end comprises a functional group for bonding to the nanoparticle core selected from a carboxylate, a sulfonate, a phosphate, and a silane.

6. The nanostructure of claim 1, wherein the PEG polymer has a negatively charged terminal functional group.

7. The nanostructure of claim 1, wherein the PEG polymer has a molecular weight ranging from between about 350 Daltons to about 5000 Daltons.

8. The nanostructure of claim 1, wherein the polyethylene imine polymer has a molecular weight ranging from between about 800 Daltons to about 1600 Daltons.

9. The nanostructure of claim 1 having a non-zero surface charge in a range from between about −50 mV to about +50 mV.

10. The nanostructure of claim 9 having a non-zero surface charge in a range from between about −25 to about +25 mV.

11. The nanostructure of claim 10 having a surface charge in a range from between about -5 mV to about −15 mV.

12. The nanostructure of claim 10 having a surface charge in a range from between about +5 mV to about +15 mV.

13. A method of making the nanostructure of claim 1 comprising:
reacting a nanoparticle core with a PEI-PEG graft having a linking group;
wherein the linking group has a functional group capable of reaction with the nanoparticle core; and
wherein said functional group is selected from the group consisting of a carboxylate, a sulfonate, a phosphate, and a trialkoxysilane.

14. The method of claim 13, wherein the nanoparticle core is superparamagnetic iron oxide.

15. A method of imaging an inflammatory condition in a mammal comprising:
introducing into the mammal the nanostructure of claim 1;
permitting the nanostructure of claim 1 to migrate to inflamed tissue; and
imaging the inflamed tissue using magnetic resonance.

16. The method of claim 15, further comprising managing the inflammatory condition.

17. The method of claim 15, wherein the mammal is a human.

18. The method of claim 15, further comprising treating the mammal to decrease inflammation before, after, or before and after imaging the inflammatory condition, and using the results to manage the inflammatory condition.

19. The method of claim 15, wherein the introducing step comprises administering the agent topically, intravascularly, intramuscularly, or interstitially.

20. The method of claim 17, wherein about 0.1 mg Fe/kg to about 50 mg Fe/kg of the nanostructure is administered to the human.

21. The method of claim 17, wherein about 0.1 mg Fe/kg to about 2.5 mg Fe/kg of the nanostructure is administered to the human.

22. The method of claim 15, wherein the inflammatory condition is associated with macrophage accumulation.

23. The method of claim 15, wherein the inflammatory condition is a condition selected from the group consisting of an autoimmune condition, a vascular condition, a neurological condition, and a combination thereof.
